(19) United States
(12) Patent Application Publication
(10) Pub. No.: US 2005/0171353 A1
(43) Pub. Date: Aug. 4, 2005

(54) Piperidine or 8-aza-bicyclo[3.2.1]oct-3-yl derivatives useful as modulators of chemokine receptor activity (especially ccr5)

(76) Inventor: John Cumming, Macclesfield (GB)

Correspondence Address: FISH & RICHARDSON P.C., 225 FRANKLIN STREET, BOSTON, MA, 02110, US

(21) Appl. No.: 10/508,895

(22) PCT Filed: Mar. 24, 2003
(86) PCT No.: PCT/SE03/00480

(30) Foreign Application Priority Data
Mar. 25, 2002 (SE) 0200919-9

(51) Int. Cl. C07D 453/02; C07D 41/02; C07D 211/56

(52) U.S. Cl. 546/135; 546/207; 546/224

(57) ABSTRACT

The invention provides a compound of formula (I) wherein A, R¹, R², R³, R^{3a}, R⁴, R^{4a}, R⁵, and R⁶ are as defined; or a pharmaceutically acceptable salt thereof or a solvate thereof; compositions containing these compounds, processes for preparing them and their use as modulators of chemokine activity (for example CCR5 activity).

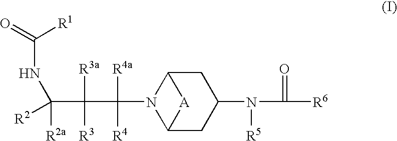

The present invention relates to piperidine derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in PCT/SE01/01053, EP-A1-1013276, WO00/08013, WO01/90106, WO99/38514 and WO99/04794.

Chemokines are chemotactic cytokines that are released by a wide variety of cells to attract macrophages, T cells, eosinophils, basophils and neutrophils to sites of inflammation and also play a role in the maturation of cells of the immune system. Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MIP-1α and MIP-1β and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I): 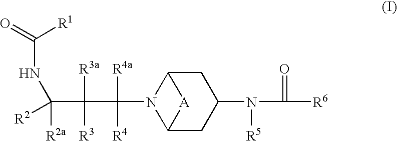
wherein:
A is CH₂CH₂ or A is absent;
R¹ is C₃₋₇ cycloalkyl (substituted by one or two fluorine atoms and optionally further substituted by C₁₋₄ alkyl) or N-linked heterocyclyl (substituted by one or two fluorine atoms and optionally further substituted by C₁₋₄ alkyl);
R² is C₃₋₆ alkyl or C₃₋₆ cycloalkyl, or phenyl or heteroaryl either of which is optionally substituted by halogen, C₁₋₄ alkyl, C₁₋₄ alkoxy, S(O)ₙ(C₁₋₄ alkyl), nitro, cyano or CF₃;
R^{2a}, R⁴ and R^{4a} are, independently, hydrogen or C₁₋₄ alkyl;
R³ and R^{3a} are, independently, hydrogen or C₁₋₄ alkyl or C₁₋₄ alkoxy;
R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;
R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH; wherein the phenyl and heteroaryl rings of R⁶ are optionally substituted by halo, cyano, nitro, hydroxy, C₁₋₄ alkyl, C₁₋₄ alkoxy, S(O)ₘC₁₋₄ alkyl, S(O)₂NR⁷R⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R⁷ and R⁸ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);
m, n and q are, independently, 0, 1 or 2;
or a pharmaceutically acceptable salt thereof or a solvate thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts (also known as adducts) such as a hydrochloride, hydrobromide, phosphate, acetate, fumarate, maleate, tartrate, citrate, oxalate, methanesulphonate or p-toluenesulphonate. An acid addition salt, is, for example, a hydrochloride.

The compounds of the invention may exist as solvates (such as hydrates) and the present invention covers all such solvates.

Alkyl groups and moieties contain, unless otherwise specified, for example 1-6, such as 1-4, carbon atoms. Alkyl groups and moieties are straight or branched chain and are, for example, methyl, ethyl, n-propyl or iso-propyl.

Alkenyl includes prop-2-en-1-yl, allyl, but-3-en-1-yl, but-1-en-1-yl or 2-methylallyl. Alkynyl includes propargyl or but-3-yn-1-yl. Alkenyl and alkynyl groups and moieties are, for example, allyl or propargyl.

Cycloalkyl contains, unless otherwise specified, for example 3-7, such as 3-6, carbon atoms. Cycloalkyl is, for example, cyclopropyl, cyclobutyl or cyclopentyl.

When A is present the central ring of formula (I) is a 3-substituted 8-aza-bicyclo[3.2.1]oct-8-yl ring. When A is absent the central ring of formula (I) is a 4-substituted piperidin-1-yl ring.

Heterocyclyl is a non-aromatic, monocyclic ring comprising at least one nitrogen, and, optionally, one further heteroatom selected from the group comprising nitrogen, oxygen and sulphur. Heterocyclyl includes aziridinyl, azetidinyl, piperidinyl, morpholinyl, thiomorpholinyl, pyrrolidinyl or piperazinyl.

Heteroaryl is an aromatic 5 or 6 membered ring comprising at least one heteroatom selected from the group comprising nitrogen, oxygen and sulphur. Heteroaryl is, for example, pyrrolyl, imidazolyl, pyrazolyl, 1,2,3-triazolyl, 1,2,4-triazolyl, oxazolyl, isoxazolyl, oxadiazolyl, thiazolyl, isothiazolyl, thiadiazolyl, pyridinyl, pyrimidinyl, pyrazinyl, pyridazinyl, thienyl, furyl, quinolinyl, isoquinolinyl, dihydroisoquinolinyl, quinazolinyl, quinoxalinyl, indolyl, isoindolyl, benzimidazolyl, benzo[b]furyl, benzo[b]thienyl, phthalazinyl, benzthiazolyl or cinnolinyl.

Phenylalkyl is, for example, benzyl, 1-(phenyl)eth-1-yl or 1-(phenyl)eth-2-yl.

Heteroarylalkyl is, for example, pyridinylmethyl, pyrimidinylmethyl or 1-(pyridinyl)eth-2-yl.

The group S(O)₂NR⁷R⁸ is, for example, S(O)₂NH₂, S(O)₂NH(C₁₋₄ alkyl), S(O)₂N(C₁₋₄ alkyl)₂, S(O)₂(4-C(O)H-piperazin-1-yl) or S(O)₂(4-C(O)CH₃-piperazin-1-yl).

Phenyl(C₁₋₂ alkyl)NH is, for example, benzylamino. Heteroaryl(C₁₋₂ alkyl)NH is, for example, pyridinylCH₂NH, pyrimidinylCH₂NH or pyridinylCH(CH₃)NH.

In one aspect the present invention provides a compound of formula (I) wherein R¹ is C₃₋₇ cycloalkyl (substituted by 1 or 2 fluorine atoms and optionally further substituted by C₁₋₄ alkyl).

In another aspect of the invention R¹ is C₃₋₇ cycloalkyl substituted by 2 fluorine atoms.

When R¹ includes a cycloalkyl ring that ring is, for example, cyclobutyl, cyclopentyl or cyclohexyl; and further the ring is, for example, cyclohexyl.

In a further aspect of the invention R¹ is 4,4-di-fluoro-cyclohexyl, 3,3-di-fluoro-cyclopentyl or 3,3-di-fluoro-cyclobutyl.

In a still further aspect of the invention R¹ is, for example, 4,4-difluorocyclohex-1-yl.

In another aspect R¹ is N-linked heterocyclyl (substituted by 1 or 2 fluorine atoms and optionally further substituted by C₁₋₄ alkyl). N-Linked heterocyclyl is, for example piperidin-1-yl or pyrrolidin-1-yl. R¹ is, for example, 4-fluoro-piperidin-1-yl or 3-fluoro-pyrrolidin-1-yl.

When R² is C₃₋₆ alkyl it is, for example, a butyl group (such as iso-butyl) and when it is C₃₋₆ cycloalkyl it is, for example, cyclopropyl or cyclohexyl.

In yet another aspect R² is phenyl or 6-membered heteroaryl optionally substituted in the ortho or meta position.

In a further aspect R² is phenyl or 6-membered heteroaryl optionally substituted (for example in the 2-, 3-, or 3- and 5-positions) by halogen or CF₃, wherein halogen is, for example, fluorine or chlorine. For example R² is 3-fluorophenyl, 3-chlorophenyl, 3-CF₃-phenyl, 4-fluorophenyl or 4-CF₃-phenyl.

In a still further aspect R² is optionally substituted (for example unsubstituted or substituted in the 3-, or 3- and 5-positions) phenyl (such as optionally substituted by halo (such as chloro or fluoro), cyano, methyl, ethyl, methoxy, ethoxy or CF₃); or R² can additionally be phenyl optionally substituted (for example unsubstituted or mono-substituted) heteroaryl (such as optionally substituted by halo (such as chloro or fluoro), cyano, methyl, ethyl, methoxy, ethoxy or CF₃).

In another aspect R² is optionally substituted (for example unsubstituted or substituted in the 3-, or 3- and 5-positions) phenyl (such as optionally substituted by halo (for example chloro or fluoro)). For example R² is phenyl, 3-fluorophenyl, 3-chlorophenyl or 3,5-difluorophenyl.

In a further aspect R^{2a}, R³, R^{3a} and R⁴ are all hydrogen.

In still further aspect R^{4a} is hydrogen or methyl. In another aspect R^{4a} is hydrogen. In a further aspect R^{4a} is methyl.

In another aspect R⁵ is hydrogen, methyl or ethyl. In yet another aspect of the invention R⁵ is ethyl.

In a further aspect R⁵ is iso-propyl.

In a still further aspect R⁵ is C₃₋₄ alkenyl, C₃₋₄ alkynyl, C₃₋₇ cycloalkyl or C₃₋₇ cycloalkyl(C₁₋₄ alkyl). For example R⁵ is allyl, propargyl, cyclopropyl or cyclopropylCH₂. In another aspect R⁵ is cyclopropyl or, for example, allyl.

In yet another aspect of the invention R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH; wherein the phenyl and heteroaryl rings of R⁶ are substituted by one of: S(O)ₘC₁₋₄ alkyl, NHC(O)NH₂, C(O)(C₁₋₄ alkyl), CHF₂, CH₂F, CH₂CF₃ or OCF₃, and optionally further substituted by one or more of halo, cyano, nitro, hydroxy, C₁₋₄ alkyl, C₁₋₄ alkoxy, S(O)ₘC₁₋₄ alkyl, S(O)₂NR⁷R⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; wherein m, R⁷ and R⁸ are hereinbefore defined.

In a still further aspect of the invention R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH (for example phenyl or phenylCH₂); wherein the phenyl and heteroaryl rings of R⁶ are substituted by S(O)₂C₁₋₄ alkyl, and optionally further substituted by one or more of halo, cyano, nitro, hydroxy, C₁₋₄ alkyl, C₁₋₄ alkoxy, S(O)ₘC₁₋₄ alkyl, S(O)₂NR⁷R⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; wherein m, R⁷ and R⁸ are hereinbefore defined.

In another aspect of the invention R⁶ is phenyl(C₁₋₂ alkyl) (for example benzyl); wherein the phenyl ring of R⁶ is substituted by S(O)₂C₁₋₄ alkyl, and optionally further substituted by one or more of halo, cyano, nitro, hydroxy, C₁₋₄ alkyl, C₁₋₄ alkoxy, S(O)ₘC₁₋₄ alkyl, S(O)₂NR⁷R⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; wherein m, R⁷ and R⁸ are hereinbefore defined.

In yet another aspect of the invention R⁶ is optionally substituted benzyl, for example benzyl singly substituted (such as in the 4-position) by S(O)₂(C₁₋₄)alkyl (such as S(O)₂CH₃) or S(O)₂NR⁷R⁸ {R⁷ and R⁸ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl)} (such as S(O)₂NH₂, S(O)₂NH(CH₃), S(O)₂N(CH₃)₂, S(O)₂(4-C(O)H-piperazin-1-yl) or S(O)₂(4-C(O)CH₃-piperazin-1-yl). The 5- or 6-membered ring is, for example, morpholine, thiomorpholine, piperidine, piperazine or pyrrolidine; such as piperazine.

In another aspect of the invention R⁶ is benzyl singly substituted (such as in the 4-position) by S(O)₂(C₁₋₄)alkyl (such as S(O)₂CH₃).

In a further aspect of the invention A is absent.

In another aspect of the invention A is CH₂CH₂.

In yet another aspect R⁷ and R⁸ are, independently, hydrogen or C₁₋₄ alkyl.

In a further aspect the compound of the invention is in free base form.

In a still further aspect the present invention provides a compound of formula (I) wherein A is absent or is CH₂CH₂; R¹ is C₃₋₆ cycloalkyl disubstituted with halo (such as fluoro), heterocyclyl monosubstituted by halo (such as fluoro); heterocyclyl is, for example, piperidinyl or pyrrolidinyl; R² is phenyl or monohalophenyl or dihalophenyl, where halo is, for example, fluoro, (for example R² is phenyl, 3-fluorophenyl or 3,5-difluorophenyl); R^{2a}, R³, R^{3a}and R⁴ are all hydrogen; R^{4a} is hydrogen or C₁₋₄ alkyl (such as methyl); R⁵ is C₁₋₄ alkyl (such as ethyl); R⁶ is benzyl singly substituted (such as in the 4-position) by S(O)₂(C₁₋₄)alkyl (such as S(O)₂CH₃); or an acid addition salt thereof (such as a hydrochloride).

In yet another aspect the present invention provides a compound of formula (Ia): 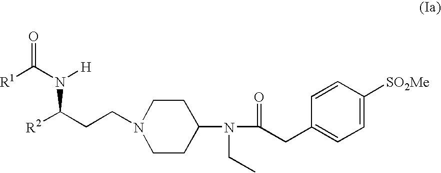
wherein R¹ and R² are as defined above, and having the absolute configuration shown.

In a further aspect the present invention provides a compound of formula (Ib): 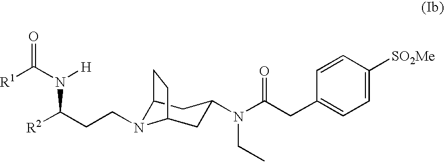
wherein R¹ and R² are as defined above, and having the absolute configuration shown.

In a still further aspect the present invention provides a compound of formula (Ic): 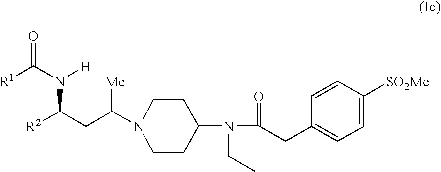
wherein R¹ and R² are as defined above, and having the absolute configuration shown.

The compounds in Tables I, II and III illustrate the invention.

TABLE I
Table I comprises compounds of the invention having the formula (Ia).

| | |
|---|---|
| | (Ia) |
| 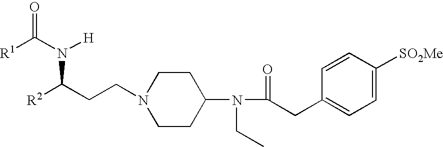 | |

| Compound | | | | LCMS |
|---|---|---|---|---|
| No. | R¹ | R² | Adduct | (MH+) |
| 1 | 4,4-difluoro- | Phenyl | | 604 |
| | cyclohexyl | | | |
| 2 | 4-fluoro- | Phenyl | | 587 |
| | piperidin-1-yl | | | |
| 3 | (R)-3-fluoro- | Phenyl | | 573 |
| | pyrrolidin-1-yl | | | |
| 4 | (S)-3-fluoro- | Phenyl | | 573 |
| | pyrrolidin-1-yl | | | |
| 5 | 4,4-difluoro- | 3-fluoro- | | 594 |
| | cyclohexyl | phenyl | | |
| 6 | 3,3-difluoro- | 3,5-difluoro- | hydrochloride | 612 |
| | cyclobutyl | phenyl | | |
| 7 | 4,4-difluoro- | 3,5-difluoro- | hydrochloride | 640 |
| | cyclohexyl | phenyl | | |
| 8 | 3,3-difluoro- | Phenyl | hydrochloride | 576 |
| | cyclobutyl | | | |
| 9 | 3,3-difluoro- | 3-fluoro- | hydrochloride | 594 |
| | cyclobutyl | phenyl | | |
| 10 | (R)-3,3-difluoro- | Phenyl | | |
| | cyclopentyl | | | |
| 11 | (S)-3,3-difluoro- | Phenyl | | |
| | cyclopentyl | | | |
| 12 | (R)-3,3-difluoro- | 3,5-difluoro- | | |
| | cyclopentyl | phenyl | | |
| 13 | (S)-3,3-diiluoro- | 3,5-difluoro- | | |
| | cyclopentyl | phenyl | | |
| 14 | (R)-3,3-difluoro- | 3-fluoro- | | |
| | cyclopentyl | phenyl | | |
| 15 | (S)-3,3-difluoro- | 3-fluoro- | | |
| | cyclopentyl | phenyl | | |

TABLE II
Table I comprises compounds of the invention having the formula (Ib).

| | |
|---|---|
| | (Ib) |
| 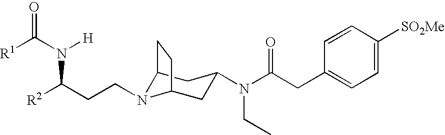 | |

| Compound | | | LCMS |
|---|---|---|---|
| No. | R¹ | R² | (MH+) |
| 1 | 4,4-difluoro-cyclohexyl | Phenyl | 604 |
| 2 | 4-fluoro-piperidin-1-yl | Phenyl | |
| 3 | (R)-3-fluoro-pyrrolidin-1-yl | Phenyl | |
| 4 | (S)-3-fluoro-pyrrolidin-1-yl | Phenyl | |
| 5 | 4,4-difluoro-cyclohexyl | 3-fluoro-phenyl | |
| 6 | 3,3-difluoro-cyclobutyl | 3,5-difluoro-phenyl | |
| 7 | 4,4-difluoro-cyclohexyl | 3,5-difluoro-phenyl | |
| 8 | 3,3-difluoro-cyclobutyl | Phenyl | |
| 9 | 3,3-difluoro-cyclobutyl | 3-fluoro-phenyl | |
| 10 | (R)-3,3-difluoro- | Phenyl | |
| | cyclopentyl | | |
| 11 | (S)-3,3-difluoro-cyclopentyl | Phenyl | |
| 12 | (R)-3,3-difluoro- | 3,5-difluoro-phenyl | |
| | cyclopentyl | | |
| 13 | (S)-3,3-difluoro-cyclopentyl | 3,5-difluoro-phenyl | |
| 14 | (R)-3,3-difluoro- | 3-fluoro-phenyl | |
| | cyclopentyl | | |
| 15 | (S)-3,3-difluoro-cyclopentyl | 3-fluoro-phenyl | |

TABLE III
Table I comprises compounds of the invention having the formula (Ic).

| | |
|---|---|
| | (Ic) |
| 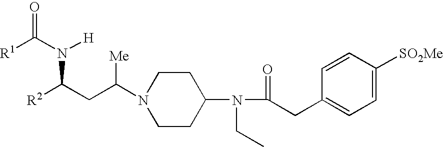 | |

| Compound | | | | LCMS |
|---|---|---|---|---|
| No. | R¹ | R² | Adduct | (MH+) |
| 1 | 4,4-difluoro- | Phenyl | hydrochloride | 618 |
| | cyclohexyl | | | |
| 2 | 4-fluoro- | Phenyl | | |
| | piperidin-1-yl | | | |
| 3 | (R)-3-fluoro- | Phenyl | | |
| | pyrrolidin-1-yl | | | |
| 4 | (S)-3-fluoro- | Phenyl | | |
| | pyrrolidin-1-yl | | | |
| 5 | 4,4-difluoro- | 3-fluoro- | | |
| | cyclohexyl | phenyl | | |
| 6 | 3,3-difluoro- | 3,5-difluoro- | | |
| | cyclobutyl | phenyl | | |
| 7 | 4,4-difluoro- | 3,5-difluoro- | | |
| | cyclohexyl | phenyl | | |
| 8 | 3,3-difluoro- | Phenyl | hydrochloride | 590 |
| | cyclobutyl | | | |
| 9 | 3,3-difluoro- | 3-fluoro- | | |
| | cyclobutyl | phenyl | | |
| 10 | (R)-3,3-difluoro- | Phenyl | | |
| | cyclopentyl | | | |
| 11 | (S)-3,3-difluoro- | Phenyl | | |
| | cyclopentyl | | | |
| 12 | (R)-3,3-difluoro- | 3,5-difluoro- | | |
| | cyclopentyl | phenyl | | |
| 13 | (S)-3,3-difluoro- | 3,5-difluoro- | | |
| | cyclopentyl | phenyl | | |
| 14 | (R)-3,3-difluoro- | 3-fluoro- | | |
| | cyclopentyl | phenyl | | |
| 15 | (S)-3,3-difluoro- | 3-fluoro- | | |
| | cyclopentyl | phenyl | | |

The compounds of formulae (I), (Ia), (Ib) and (Ic) can be prepared as described below, by adaptation of methods described in the art (such as WO 01/90106) or by following or adapting the Examples or Methods provided below.

Specifically, a compound of formula (I) or (Ia) can be prepared by treating a compound of formula (II): 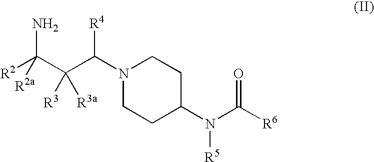
with: an acid chloride of formula R¹C(O)Cl, in the presence of a base (such as a tertiary amine, for example triethylamine) and in a suitable solvent (such as a chlorinated hydrocarbon, for example dichloromethane); or an acid of formula R¹CO₂H in the presence of a suitable coupling agent (such as O-(7-azabenzotriazol-1-yl)-N,N,N′,N′-tetramethyluronium hexafluorophosphate [HATU] or bromo-tris-pyrrolidino-phosphonium hexafluorophosphate [PyBrop]) in the presence of a suitable base (such as a tertiary amine, for example diisopropylethylamine) in a suitable solvent (such as N-methylpyrrolidinone).

A compound of formula (II) can be prepared by treating a compound of formula (III): 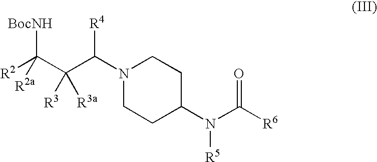
with trifluoroacetic acid or hydrochloric acid in the presence of methanol, and then basifying to release the free amine form of formula (II).

A compound of formula (III) can be prepared by reductively aminating a compound of formula (IV): 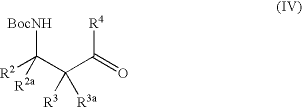
with a compound of formula (V): 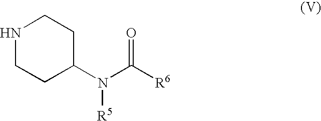
in the presence of a suitable solvent (such as an aliphatic alcohol such as methanol), a suitable organic acid (such as an aliphatic acid, for example acetic acid) and a suitable reducing agent (such as sodium triacetoxyborohydride or sodium cyanoborohydride).

A compound of formula (II) wherein R^{2a} is hydrogen can be prepared by reductive amination of a compound of formula (VI): 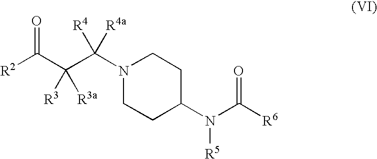
for example by reacting a compound of formula (VI) with hydroxylamine and hydrogenating the product so formed with hydrogen in the presence of a suitable metal catalyst (such as palladium or platinum catalyst, for example palladium on charcoal).

A compound of formula (VI), wherein R^{4a} is hydrogen, can be prepared by reacting a compound of formula (V) with:
an alkyl halide of formula R²C(O)CR³R^{3a}CHR⁴X (wherein X is halogen, such as chloro, bromo or iodo) in the presence of a suitable base (such as potassium carbonate) and a suitable solvent (such as acetone); or,
compounds of formula R²C(O)CHR³R^{3a} and R⁴CHO in the presence of a suitable acid (such as acetic acid).

A compound of formula (VI), wherein R^{3a} is hydrogen, can be prepared by reacting a compound of formula (V) with an alkene of formula R²C(O)CR³═CR⁴R^{4a} in a suitable solvent (such as an aliphatic alcohol, for example ethanol) at a temperature in the range −10 to 100° C.

Compounds of formula (Ib) can be prepared by referring to WO 01/90106 and WO 01/87839.

The starting materials for these processes are commercially available, can be prepared by literature methods or can be prepared by adapting literature methods. In a further aspect the invention provides processes for preparing the compounds of formulae (I), (Ia), (Ib) and (Ic). Many of the intermediates in the processes are novel and these are provided as further features of the invention.

The compounds of the invention have activity as pharmaceuticals, in particular as modulators (such as agonists, partial agonists, inverse agonists or antagonists) of chemokine receptor (for example CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)). Examples of these conditions are:
(1) (the respiratory tract) obstructive diseases of airways including: chronic obstructive pulmonary disease (COPD) (such as irreversible COPD); pulmonary fibrosis; asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;
(3) (skin and eyes) psoriasis, atopic dermatitis, contact dermatitis or other eczmatous dermitides, seborrhoetic dermatitis, lichen planus, phemphigus, bullous phemphigus, epidermolysis bullosa, urticaria, angiodermas, vasculitides erythemas, cutaneous eosinophilias, uveitis, alopecia areata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, inhibiting the entry of viruses into target cells, Acquired Immunodeficiency Syndrome (AIDS), lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura, disorders of the menstrual cycle, glomerulonephritis or cerebral malaria.

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the formula (I), (Ia), (Ib) or (Ic) (for example a compound of formula (I), (Ia) or (Ib)), or a pharmaceutically acceptable salt thereof or a solvate thereof, for use in a method of treatment of a warm blooded animal (such as man) by therapy (including prophylaxis).

According to a further feature of the present invention there is provided a method for modulating chemokine receptor activity (for example CCR5 receptor activity) in a warm blooded animal, such as man, in need of such treatment, which comprises administering to said animal an effective amount of a compound of the present invention, or a pharmaceutically acceptable salt thereof or a solvate thereof.

The present invention further provides a method of treating a chemokine mediated disease state (for example a CCR5 mediated disease state, such as rheumatoid arthritis) in a warm blooded animal (such as man) suffering from, or at risk of, said disease, which comprises administering to an animal in need of such treatment a therapeutically effective amount of a compound of formula (I), (Ia), (Ib) or (Ic) (for example a compound of formula (I), (Ia) or (Ib)), or a pharmaceutically acceptable salt thereof or solvate thereof.

The invention also provides a compound of the formula (I), (Ia), (Ib) or (Ic) (for example a compound of formula (I), (Ia) or (Ib)), or a pharmaceutically acceptable salt thereof or a solvate thereof, for use in therapy (including prophylaxis); for example in the treatment of a chemokine mediated disease state (for example a CCR5 mediated disease state) in a warm blooded animal, such as man, such as in the treatment of rheumatoid arthritis.

The invention also provides a compound of the formula (I), (Ia), (Ib) or (Ic) (for example a compound of formula (I), (Ia) or (Ib)), or a pharmaceutically acceptable salt thereof or a solvate thereof, for use as a medicament, for example a medicament for the treatment of rheumatoid arthritis.

In another aspect the present invention provides the use of a compound of the formula (I), (Ia), (Ib) or (Ic) (for example a compound of formula (I), (Ia) or (Ib)), or a pharmaceutically acceptable salt thereof or a solvate thereof, in the manufacture of a medicament for use in therapy (for example in modulating chemokine receptor activity (for example CCR5 receptor activity (for example in the treatment of rheumatoid arthritis)) in a warm blooded animal, such as man).

The invention further provides the use of a compound of formula (I), (Ia), (Ib) or (Ic) (for example a compound of formula (I), (Ia) or (Ib)), or a pharmaceutically acceptable salt thereof, in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;
(3) (skin and eyes) psoriasis, atopic dermatitis, contact dermatitis or other eczmatous dermitides, seborrhoetic dermatitis, lichen planus, phemphigus, bullous phemphigus, epidermolysis bullosa, urticaria, angiodermas, vasculitides erythemas, cutaneous eosinophilias, uveitis, alopecia areata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura or disorders of the menstrual cycle;
in a warm blooded animal, such as man.

In order to use a compound of the invention, or a pharmaceutically acceptable salt thereof or solvate thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the formula (I), (Ia), (Ib) or (Ic) (for example a compound of formula (I), (Ia) or (Ib)), or a pharmaceutically acceptable salt thereof or a solvate thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat. A suitable pharmaceutical composition of this invention is one suitable for oral administration in unit dosage form, for example a tablet or capsule which contains between 0.1 mg and 1 g of active ingredient.

Each patient may receive, for example, an intravenous, subcutaneous or intramuscular dose of 0.01 mgkg⁻¹ to 100 mgkg⁻¹ of the compound, for example in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The invention will now be illustrated by the following non-limiting Examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;
(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK. Where “Argonaut™ PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.
(iv) in general, the course of reactions was followed by TLC and reaction times are given for illustration only;
(v) yields, when given, are for illustration only and are not necessarily those which can be obtained by diligent process development; preparations were repeated if more material was required;
(vi) when given, ¹H NMR data is quoted and is in the form of delta values for major diagnostic protons, given in parts per million (ppm) relative to tetramethylsilane (TMS) as an internal standard, determined at 300 MHz using perdeuterio DMSO (CD₃SOCD₃) as the solvent unless otherwise stated; coupling constants (J) are given in Hz;
(vii) chemical symbols have their usual meanings; SI units and symbols are used;
(viii) solvent ratios are given in percentage by volume;
(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;
(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and

(xi) the following abbreviations are used:

| | |
|---|---|
| THF | tetrahydrofuran; |
| Boc | tert-butoxycarbonyl; |
| THF | tetrahydrofuran; |
| DCM | dichloromethane; and |
| HATU | O-(7-Azabenzotriazol-l-yl)-N,N,N′,N′-tetramethyluronium |
| | hexafluorophosphate. |

### EXAMPLE 1

This Example illustrates the preparation of (S)—N-[1-(3-phenyl-3-[4,4-difluorocyclohexylcarboxyamino]propyl)-4-piperidinyl]-N-ethyl-4-methanesulfonylphenylacetamide (Compound No. 1 of Table I).

(S)—N-[1-(3-Phenyl-3-aminopropyl)-4piperidinyl]-N-ethyl-4-methanesulfonylphenylacetamide dihydrochloride (Method A, 250 mg), 4,4-difluorocyclohexane carboxylic acid (100 mg) and N,N-di-isopropylethylamine (0.7 mL) were stirred in DCM (5 mL) at room temperature. To this solution was added HATU (200 mg) and stirring was continued for 16 hours. 2N Sodium hydroxide solution (2 mL) was added and the organic layer separated, washed with water and concentrated; the residue was purified by silica gel chromatography (eluent 0-30% methanol in ethyl acetate) to give the title compound as a colourless gum (110 mg); NMR: 1.0 and 1.1 (t, 3H), 1.7 (m, 7H), 2.2 (m, 6H), 3.0 (m, 3H), 3.2 (s, 3H), 3.4 (q, 2H), 3.8 and 3.9 (s, 2H), 4.1 and 4.3 (m, 1H), 4.8 (m, 1H), 7.2 (m, 1H), 7.3 (m, 4H), 7.5 (d, 2H), 7.8 (d, 2H), 8.85 (m, 1H); MS: 604 (MH+).

The procedure described in Example 1 can be repeated using different carboxylic acids (such as 3,3-di-fluorocyclobutane carboxylic acid) in place of 4,4-difluorocyclohexane carboxylic acid and different amines or amine dihydrochlorides (such as (S)—N-{1-[3-amino-3-(3-fluorophenyl)propyl]piperidin-4-yl}-N-ethyl-2-(4-methanesulfonyl-phenyl)acetamide (Method F), (S)—N-{1-[3-amino-3-(3,5-di-fluorophenyl)propyl]piperidin-4-yl}-N-ethyl-2-(4-methanesulfonyl-phenyl)acetamide dihydrochloride (Method G)) or N-[1-((4S)-4-phenyl-4-aminobut-2-yl)-4-piperidinyl]-N-ethyl-4-methanesulfonylphenylacetamide dihydrochloride (Method H)) in place of (S)—N-[1-(3-phenyl-3-aminopropyl)-4-piperidinyl]-N-ethyl-4-methanesulfonylphenylacetamide dihydrochloride.

### EXAMPLE 2

This Example illustrates the preparation of (S)—N-[1-(3-phenyl-3-[4-fluoropiperidin-1-ylcarboxyamino]propyl)-4-piperidinyl]-N-ethyl-4-methanesulfonylphenylacetamide (Compound No. 2 of Table I).

To (S)—N-[1-(3-phenyl-3-[4-nitrophenoxycarboxyamino]propyl)-4-piperidinyl]-N-ethyl-4-methanesulfonylphenylacetamide (Method C, 150 mg) in DCM (10 mL) was added 4-fluoropiperidine hydrochloride (100 mg) and N,N-di-isopropylethylamine (1 mL). The resulting mixture was stirred at room temperature for 16 hours. 2N Sodium hydroxide solution (10 mL) was added and the organic layer separated, washed with water, dried (MgSO₄) and concentrated; the residue was purified by silica gel chromatography (eluent 0-20% methanol in ethyl acetate) to give the title compound as a colourless gum (140 mg); MS: 587 (MH+).

The procedure described in Example 2 can be repeated using different amines (such as (S)-3-fluoro-pyrrolidine or (R)-3-fluoropyrrolidine) in place of 4-fluoropiperidine hydrochloride.

### EXAMPLE 3

This Example illustrates the preparation of (S)-4,4-difluoro-cyclohexanecarboxylic acid[3-(3-{ethyl-[2-(4-methanesulfonyl-phenyl)-acetyl]-amino}-8-aza-bicyclo[3.2.1]oct-8-yl-exo)-1-phenyl-propyl]-amide (Compound No. 1 of Table II).

To a solution of N-(8-aza-bicyclo[3.2.1]oct-3-yl-exo)-N-ethyl-2-(4-methanesulfonyl-phenyl)-acetamide (Method D; 98 mg, 0.28 mmol) in DCM was added (S)-3-phenyl-3-(4,4-difluorocyclohexylcarbonylamino)propanal (Method E; 165 mg, 0.56 mmol). To the resulting mixture was added sodium triacetoxyborohydride (119 mg). This was then stirred at room temperature for 18 h, washed with water, dried over MgSO₄ and concentrated. Purification was achieved by BondElut chromatography eluting with a gradient of DCM to 10% methanol and 1% 0.88 amnnonia in DCM to give the title compound (143 mg); NMR (CDCl₃): 1.1 and 1.2 (t, 3H), 1.3 (m, 1H), 1.9 (m, 19H), 2.3 (m, 1H), 2.5 (m, 1H), 3.0 (s, 3H), 3.3 (m, 4H), 3.8 (m, 2H), 3.6 and 4.4 (m, 1H), 5.0 (m, 1H), 7.2 (m, 5H), 7.4 (m, 2H), 7.9 (m, 2H); MS: 630 (MH+).

Below are presented certain NMR data for some compounds of the invention.

(S)—N-[1-(3-Phenyl-3-[(R)-3-fluoropyrrolidin-1-ylcarboxyamino]propyl)-4-piperidinyl]-N-ethyl4-methanesulfonylphenylacetamide (Compound No. 3 of Table I).

NMR (d6-DMSO, 120° C.): 1.1 (t, 3H), 1.5 (m, 2H), 1.8 (m, 2H), 1.9 (m, 2H), 2.1 (m, 2H), 2.3 (m, 2H), 2.9 (m, 2H), 3.15 (s, 3H), 3.3 (m, 2H), 3.35 (m, 2H), 3.5 (M, 2H), 3.6 (dd, 1H), 3.8 (s, 2H), 3.85 (m, 1H), 4.9 (dd, 1H), 5.3 (d, 1H), 6.25 (d, 1H), 7.2 (m, 1H), 7.3 (m, 4H), 7.55 (d, 2H), 7.85 (d, 2H).

(S)—N-[1-(3-Phenyl-3-[(S)-3-fluoropyrrolidin-1-ylcarboxyamino]propyl)-4-piperidinyl]-N-ethyl-4-methanesulfonylphenylacetamide (Compound No. 4 of Table I).

NMR (d6-DMSO, 120° C.): 1.1 (t, 3H), 1.5 (m, 2H), 1.8 (m, 2H), 1.9 (m, 2H), 2.1 (m, 2H), 2.3 (m, 2H), 2.9 (m, 2H), 3.15 (s, 3H), 3.3 (m, 2H), 3.35 (m, 2H), 3.5 (m, 2H), 3.6 (dd, 1H), 3.8 (s, 2H), 3.85 (m, 1H), 4.9 (dd, 1H), 5.3 (d, 1H), 6.25 (d, 1H), 7.2 (m, 1H), 7.3 (m, 4H), 7.55 (d, 2H), 7.85 (d, 2H).

(S)—N-[1-(3-[3,5-Difluorophenyl]-3-[3,3-difluorocyclobutylcarboxyamino]propyl)-4-piperidinyl]-N-ethyl-4-methanesulfonylphenylacetamide hydrochloride (Compound No. 6 of Table I).

NMR (d6-DMSO, 120° C.): 1.15 (t, 3H), 1.8 (m, 2H), 2.3 (m, 2H), 2.4 (m, 2H), 2.7-3.0 (m, 9H), 3.15 (s, 3H), 3.35 (q, 2H), 3.45 (m, 2H), 3.85 (s, 2H), 4.2 (br m, 1H), 5.0 (dd, 1H), 6.95 (dd, 1H), 7.1 (d, 2H), 7.5 (d, 2H), 7.85 (d, 2H), 8.45 (br s, 1H).

(S)—N-[1-(3-[3,5-Difluorophenyl]-3-[4,4-difluorocyclohexylcarboxyamino]propyl)-4-piperidinyl]-N-ethyl-4-methanesulfonylphenylacetamide hydrochloride (Compound No. 7 of Table I).

NMR (d6-DMSO, 120° C.): 1.1 (t, 3H), 1.7 (m, 8H), 2.1 (m, 2H), 2.3 (m, 2H), 2.4 (m, 4H), 3.0 (m, 3H), 3.15 (s, 3H), 3.35 (q, 2H), 3.45 (m, 2H), 3.85 (s, 2H), 4.2 (br m, 1H), 4.9 (m, 1H), 6.9 (dd, 1H), 7.05 (d, 2H), 7.5 (d, 2H), 7.85 (d, 2H), 8.3 (br s, 1H).

(S)—N-[1-(3-Phenyl-3-[3,3-difluorocyclobutylcarboxyamino]propyl)-4-piperidinyl]-N-ethyl-4-methanesulfonylphenylacetamide hydrochloride (Compound No. 8 of Table I).

NMR (d6-DMSO, 120° C.): 1.2 (t, 3H), 1.8 (m, 2H), 2.3 (m, 2H), 2.4 (m, 2H), 2.75 (m, 4H), 3.05 (m, 5H), 3.2 (s, 3H), 3.4 (q, 2H), 3.45 (m, 2H), 3.9 (s, 2H), 4.2 (br m, 1H), 5.0 (dd, 1H), 7.3 (m, 1H), 7.35 (m, 4H), 7.55 (d, 2H), 7.95 (d, 2H), 8.35 (br s, 1H).

(S)—N-[1-(3-[3-Fluorophenyl]-3-[3,3-difluorocyclobutylcarboxyamino]propyl)-4-piperidinyl]-N-ethyl-4-methanesulfonylphenylacetamide hydrochloride (Compound No. 9 of Table I).

NMR (d6-DMSO, 120° C.): 1.15 (t, 3H), 1.8 (m, 2H), 2.25 (m, 2H), 2.35 (m, 2H), 2.7-3.0 (m, 9H), 3.2 (s, 3H), 3.4 (q, 2H), 3.45 (m, 2H), 3.9 (s, 2H), 4.2 (br m, 1H), 5.0 (dd, 1H), 7.05 (m, 1H), 7.25 (m, 2H), 7.35 (m, 1H), 7.55 (d, 2H), 7.9 (d, 2H), 8.35 (br s, 1H).

N-[1-((4S)-4-Phenyl-4-[4,4-difluorocyclohexylcarboxyamino]but-2-yl)-4-piperidinyl]-N-ethyl-4-methanesulfonylphenylacetamide hydrochloride (Compound No. 1 of Table III).

NMR: 1.1 & 1.23 (t, 3H), 1.46 (d, 3H), 1.60 (m, 2H), 1.67 (m, 2H), 1.80 (m, 2H), 1.97 (m, 1H), 2.34-2.60 (m, 4H), 3.14 (m, 3H), 3.24 (s, 3H), 3.24-3.49 (m, 6H), 3.90 (m, 4H), 4.26 (m, 1H), 5.03 (m, 1H), 7.32 (m, 1H), 7.38 (m, 2H), 7.48 (m, 2H), 7.57 (m, 2H), 7.91 (dd, 2H), 8.48 (t, 1H).

N-[1-((4S)-4-Phenyl-4-[3,3-difluorocyclobutylcarboxyamino]but-2-yl)-4-piperidinyl]-N-ethyl-4-methanesulfonylphenylacetamide hydrochloride (Compound No. 8 of Table III).

NMR (d6-DMSO, 120° C.): 1.15 (t, 3H), 1.3 (d, 3H), 1.8 (m, 2H), 2.15 (m, 1H), 2.55 (m, 2H), 2.75 (m, 5H), 3.1-3.2 (m, 3H), 3.2 (s, 3H), 3.3 (m, 2H), 3.4 (q, 2H), 3.55 (m, 1H), 3.9 (s, 2H), 4.3 (br m, 1H), 5.0 (dd, 1H), 7.3 (m, 1H), 7.35 (m, 2H), 7.45 (m, 2H), 7.55 (d, 2H), 7.9 (d, 2H), 8.3 (br s, 1H).

Method A

### (S)—N-[1-(3-Phenyl-3-aminopropyl-4-piperidinyl]-N-ethyl-4-methanesulfonylphenylacetamide dihydrochloride

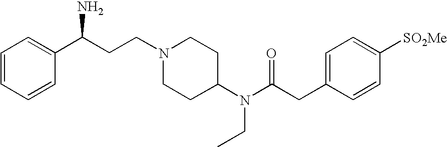

### Step 1: Preparation of 1-phenylmethyl-4-ethylaminopiperidine dihydrochloride

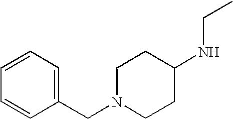

To a solution of 1-phenylmethyl-4-piperidone (25.0 g, 132 mmol) in THF (250 mL) was added ethylamine hydrochloride (12.0 g, 147 mmol) and methanol (50 mL) and the resulting mixture stirred at room temperature for 10 min. Sodium triacetoxyborohydride (40 g, 189 mmol) was added portionwise and the resulting mixture stirred at room temperature for 1 h. 2M Sodium hydroxide solution (250 mL) was added and the resulting mixture extracted with diethyl ether. The organic extracts were dried (K₂CO₃) and evaporated to give 1-phenylmethyl-4-ethylaminopiperidine as an oil. This was dissolved in ethanol (500 mL) and concentrated hydrochloric acid (20 mL) was added. The resulting crystals were collected, washed with diethyl ether and dried giving the sub-titled compound as a solid (38 g); NMR: (CDCl₃): 1.10 (t, 3H), 1.40 (m, 2H), 1.83 (m, 2H), 2.02 (m, 2H), 2.65 (q, 2H), 2.85 (m, 2H), 3.50 (s, 2H), 3.75 (m, 1H), 7.2-7.4 (m, 5H); MS: 219 (MH+).

### Step 2: Preparation of N-(1-phenylmethyl-4-piperidinyl)-N-ethyl-4-methanesulfonylphenylacetamide

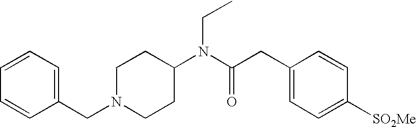

To a solution of 1-phenylmethyl-4-ethylaminopiperidine dihydrochloride (32.0 g, 110 mmol) in DCM (500 mL) was added N,N-diisopropylethylamine (60 mL) with stirring to ensure complete dissolution. 4-Methanesulfonylphenylacetic acid (25.0 g, 117 mmol), 4-Dimethylaminopyridine (4-DMAP) (2.0 g) and dicyclohexylcarbodiimide (DCCI) (25.0 g, 121 mmol) were added and the resulting mixture was stirred at room temperature for 20 h. The precipitate was removed by filtration and the resulting solution was washed successively with 2N aqueous HCl, water and 1N aqueous NaOH, dried (MgSO₄) and evaporated. The residue was purified by silica gel chromatography (eluent 10% MeOH/ethyl acetate) to afford the sub-titled compound (35 g, 76%); NMR: 1.00 and 1.14 (t, 3H), 1.45 and 1.70 (m, 2H), 1.95 (br m, 2H), 2.80 (br m, 2H), 3.18 (s, 3H), 3.20 and 3.33 (q, 2H), 3.45 (s, 2H), 3.80 and 3.87 (s, 2H), 3.70 and 4.10 (m, 1H), 7.2-7.3 (m, 5H), 7.48 (m, 2H), 7.82 (m, 2H); MS: 415 (MH+).

### Step 3: Preparation of N-(4-Piperidinyl)-N-ethyl-4-methanesulfonylphenylacetamide

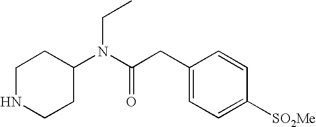

To a solution of N-(1-phenylmethyl-4-piperidinyl)-N-ethyl-4-methanesulfonylphenylacetamide (34 g, 82 mmol) in ethanol (600 mL) was added ammonium formate (40 g). The mixture was purged with argon and 30% Pd on carbon (4.2 g) was added. The resulting mixture was stirred at reflux for 4 h, then allowed to cool and filtered through diatomaceous earth. The filtrate was evaporated to give a thick oil which solidified on standing to yield the sub-titled compound (24.9 g, 94%); NMR: 1.02 and 1.15 (t, 3H), 1.4-1.6 (br m, 4H), 2.45 (m, 2H), 2.93 (br m, 2H), 3.18 (s, 3H), 3.20 and 3.32 (q, 2H), 3.72 and 4.18 (m, 1H). 3.80 and 3.87 (s, 2H), 7.50 (m, 2H), 7.85 (m, 2H); MS: 325 (MH+).

### Step 4: Preparation of Title Compound

To a solution of (S)-3-phenyl-3-Bocaminopropanal (Method B, 1.4 g, 5.6 mmol) in ethanol (100 mL) and DCM (50 mL) was added N-(4-piperidinyl)-N-ethyl-4-methanesulfonylphenylacetamide (2.0 g, 6.2 mmol), glacial acetic acid (0.6 mL, 10 mmol) and sodium triacetoxyborohydride (2.0 g, 9.4 mmol) and the resulting mixture was stirred at room temperature for 18 h. The mixture was partitioned between DCM and 2M aqueous sodium hydroxide (35 mL), and the organic phase was washed with water, dried and concentrated. The residue was suspended in methanol (10 mL) and concentrated hydrochloric acid (10 mL) was added. The resulting mixture was stirred for 30 minutes then evaporated. The residue was azeotroped with ethanol and toluene and triturated with diethyl ether yielding the title compound as a solid (1.3 g); NMR (d6 DMSO at 373K): 1.1 (t, 3H), 1.5 (m, 2H), 1.9 (m, 2H), 2.0 (m, 1H), 2.3 (m, 2H), 3.0 (m, 1H), 3.2 (m, 4H), 3.3 (q, 2H), 3.9 (s, 2H), 4.0 (m, 1H), 4.4 (m, 1H), 7.4 (m, 3H), 7.5 (m, 4H), 7.9 (m, 2H); MS: 458.

Method B

### (S)-3-Phenyl-3-Boc-aminopropanal

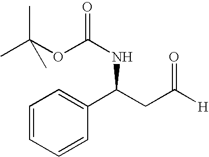

### Step 1: Preparation of (S)—N-Methyl-N-methoxy-3-phenyl-3-Bocaminopropionamide

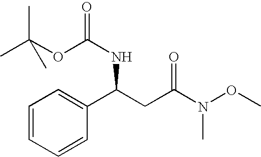

To a solution of (S)-3-phenyl-3-Bocaminopropanoic acid (available from PepTech Corp. of Cambridge, Mass., USA; 4.97 g, 18.7 mmol) in DCM (100 mL) was added DIPEA (14.8 mL, 84.8 mmol) and N,O-dimethylhydroxylamine hydrochloride (2.21 g, 22.7 mmol) followed by HATU (8.44 g, 84.8 mmol). The resulting mixture was stirred at room temperature for 18 h, diluted with DCM, washed with 2M aqueous sodium hydroxide and water. The organic phase was dried (Na₂SO₄) and concentrated. The residue was purified by silica column chromatography (eluting with isohexane then 3:1 ethyl acetate to isohexane) giving the sub-titled compound as a colourless oil (5.58 g, 97%); NMR (CDCl₃): 1.40 (s, 9H), 2.83 (dd, 1H), 3.01 (m, 1H), 3.08 (s, 3H), 3.52 (s, 3H), 5.10 (m, 1H), 7.28 (m, 5H); MS: 309.

### Step 2: Preparation of Title Compound

To a solution of (S)—N-methyl-N-methoxy-3-phenyl-3-Bocaminopropionamide (17.9 mmol) in toluene (180 mL) at −20° C. was added sodium bis(2-methoxyethoxy)aluminium hydride (65% solution in toluene, 35.8 mmol) dropwise. The resulting mixture was stirred at −15° C. for 1 h. The mixture was washed with saturated aqueous sodium dihydrogen phosphate solution (250 mL). The organic phase was dried (Na₂SO₄) and concentrated to give the title compound (5 g); NMR: 1.4 (s, 9H), 2.8 (m, 2H), 5.1 (m, 1H), 7.3 (m, 5H), 8.6 (m, 1H), 9.6 (t, 1H).

Method C

### (S)—N-[1-(3-phenyl-3-[4-nitrophenoxycarboxyamino]propyl)-4-piperidinyl]-N-ethyl-4-methanesulfonylphenylacetamide

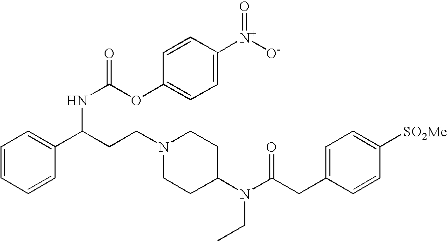

To (S)—N-[1-(3-phenyl-3-aminopropyl)-4-piperidinyl]-N-ethyl-4-methanesulfonylphenylacetamide dihydrochloride (2.0 g, 3.8 mmol) in DCM (50 mL) was added N,N-di-isopropylethylamine (2 mL) and 4-nitrophenyl chloroformate (1.0 g, 4.9 mmol) and the resulting mixture stirred at ambient temperature for 16 hours. The mixture was washed with saturated sodium bicarbonate solution (50 mL) and dried over anhydrous magnesium sulphate. The residue was purified by silica gel chromatography (eluent 0-10% methanol in ethyl acetate) to give the title compound as a pale yellow gum (2 g).

Method D

### N-(8-Aza-bicyclo[3.2.1]oct-3-yl-exo)-N-ethyl-2-(4-methanesulfonyl-phenyl)-acetamide

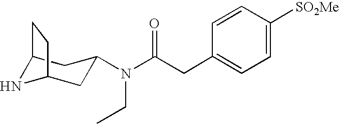

### Step 1: Preparation of N-(8-Benzyl-8-aza-bicyclo[3.2.1oct-3-yl)-N-ethyl-2-(4-methanesulfonyl-phenyl)-acetamide

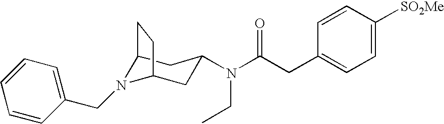

To a solution of 8-Benzyl-8-aza-bicyclo[3.2.1]oct-3-yl-exo-amine (John S. Kiely, Marland P. Hutt, Townley P. Culbertson, Ruth A. Bucsh and Donald F. Worth; J. Med. Chem., 1991, 34, 656; 2.81 g, 13 mmol) in DCM (40 mL) was added acetaldehyde (0.69 g, 16 mmol) and the resulting mixture stirred at room temperature for 1 h. Sodium triacetoxyborohydride (3.3 g, 16 mmol) was added portionwise and the resulting mixture stirred at room temperature for 16 h. The mixture was then washed with water, dried over MgSO₄ and concentrated. This material was then dissolved in DCM (50 mL) and 4-methanesulfonylphenylacetic acid (3.1 g, 14 mmol) and diisopropylcarbodiimide (2.1 g, 14 mmol) were added and the resulting mixture stirred for 2 h. The precipitate was removed by filtration and the crude material was adsorbed onto silica. Silica gel chromatography (eluent: 100% DCM to 10% methanol and 1% 0.88 ammonia in DCM) gave the subtitled compound as a foam (0.37 g); NMR (CDCl₃): 1.2 and 1.3 (t, 3H), 1.4 (m, 1H), 1.5 (m, 1H), 1.7 (m, 2H), 1.9 (m, 2H), 2.0 (m, 2H), 3.0 (s, 3H), 3.3 (m, 4H), 3.5 (d, 2H), 3.8 (d, 2H), 3.9 and 4.8 (m, 1H), 7.3 (m, 5H), 7.5 (m, 2H), 7.9 (m, 2H); MS: 441 (MH+).

### Step 2: Preparation of Title Compound

To a solution of N-(8-Benzyl-8-aza-bicyclo[3.2.1]oct-3-yl-exo)-N-ethyl-2-(4-methanesulfonyl-phenyl)-acetamide (0.37 g, 0.85 mmol) in ethanol (20 mL) was added 20% palladium hydroxide on carbon (0.04 g) and the resulting mixture was stirred under an atmosphere of hydrogen for 2 days. The catalyst was removed by filtration and the resulting solution was adsorbed onto silica. The residue was purified by silica gel chromatography (eluent: DCM to 10% methanol and 1% 0.88 ammonia in DCM) to afford the sub-titled compound as an oil(0.1 g); NMR (CDCl₃): 1.1 and 1.2 (t, 3H), 1.3 (m, 1H), 1.4 (m, 2H), 1.7 (m, 5H), 2.1 (br s, 1H), 3.0 (s, 3H), 3.3 (m, 2H), 3.6 (m, 2H), 3.7 and 3.8 (s, 2H), 3.8 and 4.8 (m, 1H), 7.4 (m, 2H), 7.9 (m, 2H); MS: 351 (MH+).

Method E

### (S)-3-Phenyl-3-(4,4-difluorocyclohexylcarbonylamino)propanal

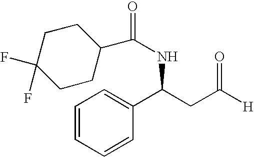

### Step 1: Preparation of (S)-3-amino-3-phenyl-propionic acid methyl ester hydrochloride

To a solution of (S)-3-Bocamino-3-phenyl-propionic acid (5 g, 18.8 mmol) in methanol (50 mL) was added thionyl chloride (1.5 mL, 20.7 mmol) dropwise. The resulting mixture was stirred at reflux for 4 h then allowed to cool and concentrated. The residue was used directly in the next reaction.

### Step 2: Preparation (S)-3-phenyl-3-(4,4-difluorocyclohexylcarbonylamino)propanol

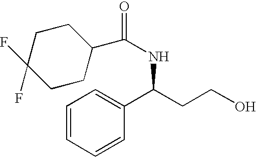

To a solution of (S)-3-amino-3-phenyl-propionic acid methyl ester hydrochloride (3.31 g, 15.3 mmol) in DCM (50 mL) was added triethylamine (1.71 g, 17 mmol) and the resulting mixture stirred at 0° C. for 10 min. Then 4,4-difluorocyclohexane carboxylic acid (2.8 g, 17 mmol) and diisopropylcarbodiimide (2.5 g, 17 mmol) were added portionwise and the resulting mixture stirred at room temperature for 16 h. The mixture was then washed with water, dried over MgSO₄ and concentrated. Silica gel chromatography (eluent: isohexane to diethyl ether) gave the sub-titled compound as a solid (3.7 g). This material was then dissolved in THF under an atmosphere of argon and lithium aluminium hydride (11 mL, 1M in THF) was added dropwise at 0° C. After stirring for 15 min, the reaction was quenched with 2M NaOH and separated. The organic layer was dried over MgSO₄ purified by silica gel chromatography (eluent: isohexane to ethyl acetate) to afford the sub-titled compound as a solid (1.32 g); NMR (CDCl₃): 1.8 (m, 8H), 2.2 (m, 3H), 3.6 (m, 1H), 3.7 (m, 1H), 5.2 (m, 1H), 7.3 (m, 5H); MS: 297 (M+).

### Step 3: Preparation of Title Compound

To a solution of (S)-3-phenyl-3-(4,4-difluorocyclohexylcarbonylamino)propanol (0.17 g, 0.56 mmol) in DCM (5 mL) was added Dess Martin periodinane (0.26 g, 0.62 mmol) and the resulting mixture was stirred for 1 h. The mixture was then washed with 2M NaOH, dried over MgSO₄ and concentrated. The resulting residue was then used directly in the preparation of Example 3.

Method F

### (S)—N-{1-[3-Amino-3-(3-fluorophenyl)propyl]piperidin-4-yl }-N-ethyl-2-(4-methanesulfonyl-phenyl)acetamide

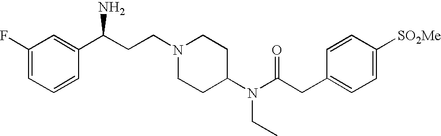

### Step 1: Preparation of trans-3-fluorocinnamic acid tert-butyl ester

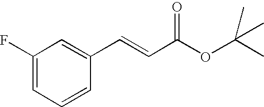

To a stirred solution of trans-3-fluorocinnamic acid (4.34 g, 26.1 mmol) in toluene (40 mL) at 110° C. was added N,N-dimethylformamide di-tert-butyl acetal (25 mL, 104 mmol) dropwise over 30 min. The resulting mixture was stirred at reflux for a further 4 h. The mixture was then cooled to room temperature and washed with water (50 mL), saturated aqueous sodium hydrogen carbonate solution (2×100 mL) and brine (100 mL), dried (MgSO₄) and evaporated. The crude product was purified by Bond Elut (isohexane then 2% ethyl acetate in isohexane) to give the title compound as a liquid (3.7 g, 64%).

### Step 2: Preparation of (S)-3-[(R)-benzyl-(1-phenyl-ethyl)-amino]-3-(3-fluoro-phenyl)-propionic acid tert-butyl ester

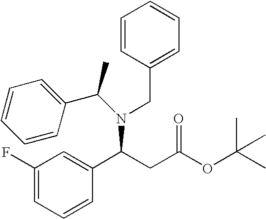

To a stirred solution of (R)-(+)-N-benzyl-α-methylbenzylamine (4.0 mL, 19 mmol) in THF (20 mL) at −78° C. was added n-butyl lithium (1.6M in hexanes, 12.5 mL, 20 mmol) and the resulting mixture was allowed to warn to room temperature over 10 min. before re-cooling to −78° C. A solution of trans-3-fluorocinnamic acid tert-butyl ester (3.74 g, 16.8 mmol) in THF (20 mL) was added and the resulting mixture was stirred at −78° C. for 2 h then quenched by the addition of saturated aqueous ammonium chloride solution (25 mL). After warming to room temperature the organic phase was washed with water (2×50 mL) and brine, dried (MgSO₄) and evaporated. The crude product was purified by Bond Elut (isohexane then 2% ethyl acetate in isohexane) to give the title compound as a gum (5.85 g, 80%); NMR (400 MHz, CDCl₃): 1.23 (s, 9H), 1.27 (d, 3H), 2.48 (m, 2H), 3.67 (s, 2H), 3.97 (q, 1H), 4.40 (dd, 1H), 6.93 (ddd, 1H), 7.1-7.4 (m, 13H).

### Step 3: Preparation of 3-tert-butoxycarbonylamino-3-(3-fluoro-phenyl)-propionic acid tert-butyl ester

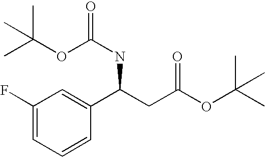

A stirred mixture of (S)-3-[(R)-benzyl-(1-phenyl-ethyl)-amino]-3-(3-fluoro-phenyl)-propionic acid tert-butyl ester (5.39 g, 12.4 mmol), di-tert-butyl dicarbonate (2.98 g, 13.7 mmol) and 20% palladium hydroxide on carbon (0.59 g) in ethanol (100 mL) was hydrogenated at 5 Bar at room temperature for 24 h. The catalyst was removed by filtration through a pad of Celite® washing through with ethanol. The filtrate was evaporated to give an oil which was partitioned between ethyl acetate and saturated aqueous sodium hydrogen carbonate solution. The organic phase was dried (MgSO₄) and evaporated. The crude product was purified by Bond Elut (eluting with isohexane then 5% ethyl acetate in isohexane) to give the title compound as an oil (3.63 g, 86%); NMR: 1.33 (s, 18H), 2.63 (m, 2H), 4.90 (m, 1H), 7.06 (ddd, 1H), 7.24 (m, 2H), 7.37 (dd, 1H), 7.50 (br d, 1H).

### Step 4: Preparation of (S)-[1-(3-fluoro-phenyl)-3-hydroxy-propyl]-carbamic acid tert-butyl ester

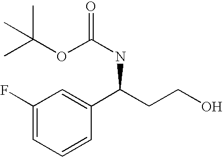

To a stirred, ice-cooled solution of 3-tert-butoxycarbonylamino-3-(3-fluoro-phenyl)-propionic acid tert-butyl ester (2.46 g, 7.25 mmol) in THF (35 mL) was added lithium aluminium hydride (1M in THF, 7.50 mL, 7.50 mmol) dropwise over 20 min. The resulting mixture was stirred with warming to room temperature for 2 h. The reaction was quenched with water (0.275 mL) then 15% aqueous sodium hydroxide (0.275 mL) and more water (0.825 mL) were added with stirring. The resultant precipitate was removed by filtration washing with THF, and the filtrate was dried (MgSO₄) and evaporated. The crude product was purified by Bond Elut (gradient elution, isohexane to 30% ethyl acetate in isohexane) to give the title compound as an oil (1.26 g, 65%); NMR: 1.4 (s, 9H), 1.75 (m, 1H), 1.85 (m, 1H), 3.3 (m, 1H), 3.4 (m, 1H), 4.5 (dd, 1H), 4.65 (br m, 1H), 7.1 (m+br s, 3H), 7.35 (m, 2H).

### Step 5: Preparation of (S)-[1-(3-fluoro-phenyl)-3-oxo-propyl]-carbamic acid tert-butyl ester

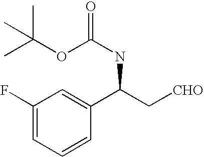

To a solution of (S)-[1-(3-fluoro-phenyl)-3-hydroxy-propyl]-carbamic acid tert-butyl ester (0.85 g, 3.2 mmol) in DCM (70 mL) under argon was added Dess-Martin periodinane (1.48 g, 3.5 mmol) and the resulting mixture was stirred at room temperature for 2 h before the addition of 2M aqueous sodium hydroxide (50 mL). The organic layer was dried (MgSO₄) and evaporated to give the title compound (quantitative); NMR: 1.4 (s, 9H), 2.8 (m, 2H), 5.1 (m, 1H), 7.05 (ddd, 1H), 7.15 (m, 2H), 7.35 (m, 1H), 7.5 (br d, 1H), 9.6 (s, 1H).

### Step 6: Preparation of (S)-[3-(4-{ethyl-[2-(4-methanesulfonyl-phenyl)-acetyl]-amino}-piperidin-1-yl)-1-(3-fluoro-phenyl)-propyl]-carbamic acid tert-butyl ester

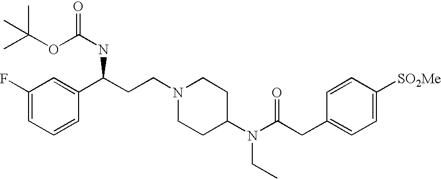

To a solution of (S)-[1-(3-fluoro-phenyl)-3-oxo-propyl]-carbamic acid tert-butyl ester (0.85 g, 3.12 mmol) in DCM (70 mL) and N-ethyl-2-(4-methanesulfonyl-phenyl)-N-piperidin-4-yl-acetamide (Method A, 1.19 g, 3.67 mmol) was added glacial acetic acid (one drop) and the resulting mixture was stirred at room temperature for 1 h. Sodium triacetoxyborohydride (1.4 g, 6.4 mmol) was added and the resulting mixture was stirred at room temperature for 18 h. The reaction mixture was quenched with water and the organic phase was washed with sodium hydrogen carbonated solution (saturated aqueous) and water, dried (MgSO₄) and concentrated. The crude product was purified by Bond Elut (ethyl acetate then 8% methanol in ethyl acetate) to give the title compound as a solid (1.00 g, 55%); NMR: 1.0 and 1.1 (t, 3H), 1.35 (s, 9H), 1.5 (m, 2H), 1.7 (m, 4H), 1.9 (m, 2H), 2.2 (t, 2H), 2.8 (m, 2H), 3.2 (s, 3H), 3.2 and 3.3 (q, 2H), 3.6 and 4.1 (m, 1H), 3.8 and 3.85 (s, 2H), 4.5 (m, 1H), 7.05 (m, 1H), 7.1 (m, 2H), 7.35 (dd, 1H), 7.5 (br d, 1H), 7.5 (d, 2H), 7.85 (d, 2H); LCMS: 576 (MH+).

### Step 7: Preparation of Title Compound

To a solution of (S)-[3-(4-{ethyl-[2-(4-methanesulfonyl-phenyl)-acetyl]-amino}-piperidin-1-yl)-1-(3-fluoro-phenyl)-propyl]-carbamic acid tert-butyl ester (1.00 g, 1.74 mmol) in THF (30 mL) and water (0.1 mL) was added trifluoroacetic acid (5.0 mL) and the resulting mixture was stirred at room temperature for 18 h. The mixture was evaporated and the residue dissolved in DCM. This solution was washed with 2M aqueous sodium hydroxide, dried (MgSO₄) and evaporated to give the title compound (0.84 g, quantitative); NMR: 1.05 and 1.09 (t, 3H), 1.45 and 1.50 (m, 2H), 1.75 (m, 4H), 1.95 (m, 2H), 2.25 (m, 2H), 2.88 (m, 2H), 3.20 (s, 3H), 3.25 and 3.30 (q, 2H), 3.67 and 4.08 (m, 1H), 3.82 and 3.89 (s, 2H), 7.00 (m, 1H), 7.15-7.40 (m, 3H), 7.50 (d, 2H); 7.85 (d, 2H), 8.70 (dd, 1H); MS: 476 (MH+).

Method G

### (S)—N-{1-[3-Amino-3-(3,5-di-fluorophenyl)propyl]piperidin-4-yl}-N-ethyl-2-(4-methanesulfonyl-phenyl)acetamide dihydrochloride

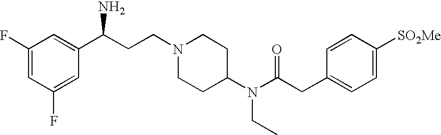

This was prepared from trans-3,5-di-fluorocinnamic acid using a similar sequence of reactions to that used to prepare (S)—N-{1-[3-amino-3-(3-fluorophenyl)propyl]piperidin-4-yl}-N-ethyl-2-(4-methanesulfonyl-phenyl)acetamide from trans-3-fluorocinnamic acid (Method F) except that the final partitioning between DCM and 2M aqueous sodium hydroxide was omitted.

Method H

### N-[1-((4S)-4-Phenyl-4-aminobut-2-yl)-4-piperidinyl]-N-ethyl-4-methanesulfonylphenylacetamide dihydrochloride

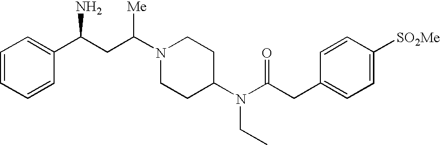

### Step 1: Preparation of (S)-4-phenyl-4-Boc-aminobutan-2-one

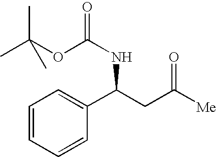

To a solution of (S)—N-methyl-N-methoxy-3-phenyl-3-Boc-aminopropionamide (Step 1 of Method B, 2.02 g, 6.56 mmol) in THF (70 mL) at −78° C. was added methylmagnesium chloride (3M in THF, 21.1 mmol) dropwise. The resulting mixture was stirred at −78° C. for 30 min. before warming to room temperature over 3 h. The reacton mixture was added to a vigorously stirred mixture of diethyl ether, ice and 1M aqueous potassium dihydrogen phosphate. The aqueous phase was extracted twice with diethyl ether and the combined organic phases washed with sodium hydrogen carbonate solution (sat. aq.) and brine, dried (Na₂SO₄) and concentrated giving the title compound as a white solid (1.27 g, 74%); NMR (CDCl₃): 1.41 (s, 9H), 2.09 (s, 3H), 2.91 (dd, 1H), 3.03 (m, 1H), 5.08 (m, 1H), 5.37 (br s, 1H), 7.28 (m, 5H); MS: 264.

### Step 2: Preparation of N-[1-((4S)-4-phenyl-4-Bocaminobut-2-yl)-4-piperidinyl]-N-ethyl-4-methanesulfonylphenylacetamide

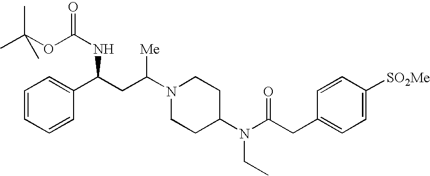

To a solution of (S)-4-phenyl-4-Boc-aminobutan-2-one (1.25 g, 4.75 mmol) and N-(4-piperidinyl)-N-ethyl-4-methanesulfonylphenylacetamide (1.54 g, 4.75 mmol) in THF/1,2-dichloroethane (1:1, 45 mL) was added titanium tetraisopropoxide (3.1 mL, 10.45 mmol) at room temperature. The resulting mixture was stirred for 15 min. before the addition of sodium triacetoxyborohydride (1.51 g, 7.11 mmol). The resulting mixture was stirred for 18 h before addition of 2M aqueous sodium hydroxide (30 mL). The mixture was diluted with DCM, filtered through Celite®, washed with brine, dried (Na₂SO₄) and concentrated. The residue was purified by BondElut chromatography eluting with a mixture of 1% methanol and 0.05% ammonia in ethyl acetate giving the title compound as a white solid (1.04 g); MS: 572.

### Step 3: Preparation of Title Compound

To N-[1-((4S)-4-phenyl-4-Bocaminobut-2-yl)-4-piperidinyl]-N-ethyl-4-methanesulfonylphenylacetamide (194 mg, 0.339 mmol) was added 5M HCl in methanol (5 mL) and the resulting mixture stirred at room temperature for 3 h. The mixture was evaporated and the residue azeotroped with toluene and triturated with diethyl ether to give the title compound as a white solid (178 mg, 98%); MS: 472.

Many intermediates are known in the art, for example 3,3-di-fluoro-cyclobutane carboxylic acid {William R. Dolbier and Dheya M. Al-Fekri; J. Org. Chem. 52, 1872-1874 (1987)}; (S)-3-fluoro-pyrrolidine and (R)-3-fluoro-pyrrolidine {Giuseppe Giardina, Giulio Dondio and Mario Grugni; SYNLETT (1995), 55-57}; and, 4,4-di-fluoro-cylohexane carboxylic acid {Mackenzie A R; Marchington A P; Middleton D S; Meadows S D; WO 97/27185-A1}.

### EXAMPLE 4

The ability of compounds to inhibit the binding of RANTES or MIP-1α was assessed by an in vitro radioligand binding assay. Membranes were prepared from Chinese hamster ovary cells which expressed the recombinant human CCR5 receptor. These membranes were incubated with 0.1 nM iodinated RANTES or MIP-1α, scintillation proximity beads and various concentrations of the compounds of the invention in 96-well plates. The amount of iodinated RANTES or MIP-1α bound to the receptor was determined by scintillation counting. Competition curves were obtained for compounds and the concentration of compound which displaced 50% of bound iodinated RANTES or MIP-1α was calculated (IC₅₀). Certain compounds of formula (I) had an IC₅₀ of less than 50 μM.

Results from this test for certain compounds of the invention are presented in Table IV. In Table IV the results are presented as Pic50 values. A Pic50 value is the negative log (to base 10) of the IC₅₀ result, so an IC50 of 1 μM (that is 1×10⁻⁶M) gives a Pic50 of 6. If a compound was tested more than once then the data below is an average of the probative tests results.

TABLE IV

| Table Number | Compound number | Pic50 |
|---|---|---|
| 1 | 1 | 8.95 |
| 1 | 2 | 7.68 |
| 1 | 3 | 7.76 |
| 1 | 6 | 8.65 |
| 2 | 1 | 8.48 |
| 3 | 8 | 9.15 |

1. A compound of formula (I): 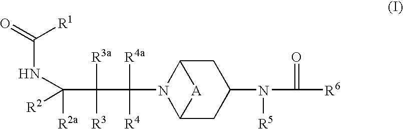
wherein:
A is CH₂CH₂ or A is absent;
R¹ is C₃₋₇ cycloalkyl (substituted by one or two fluorine atoms and optionally further substituted by C₁₋₄ alkyl) or N-linked heterocyclyl (substituted by one or two fluorine atoms and optionally further substituted by C₁₋₄ alkyl);
R² is C₃₋₆ alkyl or C₃₋₆ cycloalkyl, or phenyl or heteroaryl either of which is optionally substituted by halogen, C₁₋₄ alkyl, C₁₋₄ alkoxy, S(O)ₙ(C₁₋₄ alkyl), nitro, cyano or CF₃;
R^{2a}, R⁴ and R^{4a} are, independently, hydrogen or C₁₋₄ alkyl;
R³ and R^{3a} are, independently, hydrogen or C₁₋₄ alkyl or C₁₋₄ alkoxy;
R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;
R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH;
wherein the phenyl and heteroaryl rings of any of the foregoing are, unless stated otherwise, independently optionally substituted by halo, cyano, nitro, hydroxy, C₁₋₄ alkyl, C₁₋₄ alkoxy, S(O)ₘC₁₋₄ alkyl, S(O)₂NR⁷R⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R⁷ and R⁸ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);
m, n and q are, independently, 0, 1 or 2;
or a pharmaceutically acceptable salt thereof or a solvate thereof.

2. A compound as claimed in claim 1 wherein R^{2a}, R³, R^{3a} and R⁴ are all hydrogen.

3. A compound as claimed in claim 1 wherein R^{4a} is hydrogen or methyl.

4. A compound as claimed in claim 1 wherein R¹ is C₃₋₇ cycloalkyl (substituted by 1 or 2 fluorine atoms and optionally further substituted by C₁₋₄ alkyl).

5. A compound as claimed in claim 1 wherein R¹ is 4,4-di-fluoro-cyclohexyl, 3,3-di-fluoro-cyclopentyl or 3,3-di-fluoro-cyclobutyl.

6. A compound as claimed in claim 1 wherein R² is phenyl or 6-membered heteroaryl optionally substituted by halogen or CF₃.

7. A compound as claimed in claim 1 wherein R⁵ is ethyl.

8. A compound as claimed in claim 1 wherein R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH (for example phenyl or phenylCH₂); wherein the phenyl and heteroaryl rings of R⁶ are substituted by S(O)₂C₁₋₄ alkyl, and optionally further substituted by one or more of halo, cyano, nitro, hydroxy, C₁₋₄ alkyl, C₁₋₄ alkoxy, S(O)ₘC₁₋₄ alkyl, S(O)₂NR⁷R⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; wherein m, R⁷ and R⁸ are as defined in claim 1.

9. A process for the preparation of a compound of formula (I) as claimed in claim 1, wherein A is absent, comprising treating a compound of formula (II): 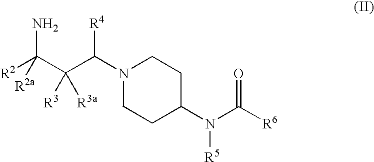
with:
an acid chloride of formula R¹C(O)Cl, in the presence of a base and in a suitable solvent; or,
an acid of formula R¹CO₂H, in the presence of a suitable coupling agent, a suitable base and in a suitable solvent.

10. A pharmaceutical composition which comprises a compound of the formula (I), or a pharmaceutically acceptable salt thereof or solvate thereof as claimed in claim 1, and a pharmaceutically acceptable adjuvant, diluent or carrier.

11-12. (canceled)

13. A method, comprising:
treating a chemokine mediated disease state in a warm blooded animal suffering from, or at risk of, said disease, which comprises administering to an animal in need of such treatment a therapeutically effective amount of a compound of formula (I), or a pharmaceutically acceptable salt thereof or solvate thereof as claimed in claim 1.
